(19) United States
(12) Patent Application Publication
(10) Pub. No.: US 2024/0099883 A1
(43) Pub. Date: Mar. 28, 2024

(54) DEVICE FOR MOBILIZING LENS MATERIAL AND POLISHING THE CAPSULAR BAG (INCLUDING AT LENS EQUATOR) DURING CATARACT SURGERY

(71) Applicant: The Regents of the University of California, Oakland, CA (US)

(72) Inventors: Jean Pierre HUBSCHMAN, Los Angeles, CA (US); Matthew J. GERBER, Los Angeles, CA (US); Tsu-Chin TSAO, Los Angeles, CA (US); Jacob ROSEN, Los Angeles, CA (US); Conor McGARTOLL, Los Angeles, CA (US); Zhantao SONG, Los Angeles, CA (US)

(73) Assignee: The Regents of the University of California, Oakland, CA (US)

(21) Appl. No.: 18/263,520

(22) PCT Filed: Jan. 28, 2022
(86) PCT No.: PCT/US2022/014442

§ 371 Date: Jul. 28, 2023

Related U.S. Application Data
(60) Provisional application No. 63/143,336, filed on Jan. 29, 2021.

(51) Int. Cl. A61F 9/007

(52) U.S. Cl. CPC A61F 9/00736; A61B 2017/320012

(57) ABSTRACT

According to certain general aspects, the present embodiments relate generally to a device that mobilizes the lens material inside the capsular bag. Mobilized lens material is easier to visualize than lens material lingering at the lens equator of the eye, or other areas in the capsular bag that are difficult to visualize, making the mobilized lens material easier to retrieve from the capsular bag. Mobilizing the lens material reduces the possibility that the person receiving the cataract surgery will develop secondary cataracts or infections due to lens material left inside the capsular bag.

### CROSS-REFERENCE TO RELATED APPLICATIONS

The present application claims priority to U.S. Provisional Patent Application No. 63/143,336 filed Jan. 29, 2021, the contents of which are incorporated herein by reference in their entirety.

### STATEMENT REGARDING FEDERALLY SPONSORED RESEARCH OR DEVELOPMENT

This invention was made with government support under Grant Number EY030595, awarded by the National Institutes of Health. The government has certain rights in the invention.

### TECHNICAL FIELD

The present embodiments relate generally to devices used in cataract surgery, and more particularly to a device that mobilizes lens material that may be hidden behind the iris or otherwise not visible during cataract surgery.

### BACKGROUND

Cataract surgery is a common surgery that is performed when cataracts cause a person's eye lens to become cloudy. The lens is the part of the eye that is responsible for focusing light necessary to create clear images of objects at various distances. The lens is located inside the capsular bag, which is behind the iris and the cornea. During cataract surgery, an incision is made in the cornea and the cataract may either be removed in its entirety, or broken up via an ultrasonic probe or a laser. The cataract is comprised of two components: the nucleus, a rigid central component, and the cortical material, or the softer outer material of the cloudy lens. If the lens is broken during surgery, the pieces of the lens must manually be removed via suction or irrigation and aspiration. Completely removing the lens pieces of the eye reduces the likelihood of secondary cataracts. Secondary cataracts may form after a person has undergone cataract surgery and can again impair a person's vision.

Although removing the lens material inside the capsular bag is desirable for a successful and complete recovery from cataract surgery, sometimes lens material remains inside the capsular bag afterwards. This can occur because the capsular bag is very delicate and translucent. If the bag breaks during cataract surgery, serious problems such as infections and retinal detachment may occur. Thus, surgeons performing cataract surgery may be forced to leave lens material inside the capsular bag to avoid fishing for lens material in the bag and inadvertently breaking the bag.

Alternatively, surgeons performing cataract surgery may believe they have cleared the capsular bag of all lens material, unknowingly leaving lens material behind the iris because the iris blocks the surgeon's complete view of the capsular bag. In particular, the equatorial region of the capsular bag is hidden by the iris. There is no known imaging technology able to penetrate the opaque iris such that the surgeon can see through the iris and into the obscured (i.e. equatorial or outer) region of the capsular bag. Therefore, technological problems remain, for example in connection with removing all of the lens material carefully and without direct human visualization. It is against this backdrop that a technological solution was sought to remedy this technological problem, among others.

### SUMMARY

According to certain aspects, the present embodiments relate generally to a device that mobilizes the lens material inside the capsular bag. Mobilized lens material is easier to visualize than lens material lingering at the lens equator of the eye, or other areas in the capsular bag that are difficult to visualize, making the mobilized lens material easier to retrieve from the capsular bag. Mobilizing the lens material reduces the possibility that the person undergoing the cataract surgery will develop secondary cataracts or infections due to lens material left inside the capsular bag.

### BRIEF DESCRIPTION OF THE DRAWINGS

These and other aspects and features of the present embodiments will become apparent to those ordinarily skilled in the art upon review of the following description of specific embodiments in conjunction with the accompanying figures, wherein:

FIG. 1A is a diagram of the side view of the anatomy of an eye.

FIG. 1B is a diagram of the top view of the anatomy of an eye.

FIG. 2A illustrates an exemplary embodiment of a tool used to mobilize cortical material from the capsular bag according to embodiments.

FIG. 2B illustrates an example prototype tool used to mobilize cortical material from the capsular bag, according to an embodiment.

FIG. 3A is a diagram of one embodiment of a tool inside the capsular bag.

FIG. 3B is a diagram of one embodiment of an activated tool inside the capsular bag.

FIG. 3C illustrates an example of emulsified lens material using a flexible loop, according to an embodiment.

FIG. 4A is a diagram of an alternate embodiment of a tool inside the capsular bag.

FIG. 4B is a diagram of an alternate embodiment of an activated tool inside the capsular bag.

FIG. 5A is a diagram of one embodiment of a retracted compliant mechanism inside the shaft of a tool.

FIG. 5B is a diagram of one embodiment of an extended compliant mechanism.

FIG. 6A is a diagram of one embodiment of sensing using the end-effector in the capsular bag.

FIG. 6B is a diagram of an alternate embodiment of sensing using the end-effector in the capsular bag.

FIG. 7 is a flowchart illustrating exemplary methodology of using a tool according to embodiments.

FIG. 8 illustrates an example handheld device having interchangeable tips according to embodiments.

FIG. 9 illustrates example aspects of robotic actuation/control of a device according to embodiments.

FIGS. 10(a), 10(b), 10(c) and 10(d) illustrates several example actuation methods of an end effector when inside the eye according to embodiments.

FIG. 11 illustrates example aspects of a straight-tip variation of a device according to embodiments.

FIG. 12 illustrates an example compliant-mechanism-based design using a motor for actuating the spinning motion of the end effector and a handheld finger press to change the end effector size according to embodiments.

FIG. 13 illustrates an example mechanical-linkage-based design that uses a motor for actuating the spinning motion of the end effector and a handheld finger press on a linkage to change the end effector size according to embodiments.

FIG. 14 illustrates an example mechanical-linkage-based design that uses a motor for actuating the spinning motion of the end effector and a handheld finger press on a linkage to change the end effector size according to embodiments.

### DETAILED DESCRIPTION

The present embodiments will now be described in detail with reference to the drawings, which are provided as illustrative examples of the embodiments so as to enable those skilled in the art to practice the embodiments and alternatives apparent to those skilled in the art. Notably, the figures and examples below are not meant to limit the scope of the present embodiments to a single embodiment, but other embodiments are possible by way of interchange of some or all of the described or illustrated elements. Moreover, where certain elements of the present embodiments can be partially or fully implemented using known components, only those portions of such known components that are necessary for an understanding of the present embodiments will be described, and detailed descriptions of other portions of such known components will be omitted so as not to obscure the present embodiments. Embodiments described as being implemented in software should not be limited thereto, but can include embodiments implemented in hardware, or combinations of software and hardware, and vice-versa, as will be apparent to those skilled in the art, unless otherwise specified herein. In the present specification, an embodiment showing a singular component should not be considered limiting; rather, the present disclosure is intended to encompass other embodiments including a plurality of the same component, and vice-versa, unless explicitly stated otherwise herein. Moreover, applicants do not intend for any term in the specification or claims to be ascribed an uncommon or special meaning unless explicitly set forth as such. Further, the present embodiments encompass present and future known equivalents to the known components referred to herein by way of illustration.

According to certain aspects, the present embodiments are related to improving cataract surgery by mobilizing lens material inside the capsular bag. While lens material is described, lens material may include, but is not limited to, a nucleus, cortical material and/or lens epithelial cells. The lens material can be mobilized along the anterior, posterior, and equatorial parts of the capsular bag.

Among other things, the present Applicant recognizes that the anatomy of a human eye makes removing lens material from the capsular bag difficult because surgeons performing cataract surgery may not see the cortical material and the lens epithelial cells, or may not be able to remove the lens epithelial cells and cortical material completely without a high risk of rupturing the capsular bag.

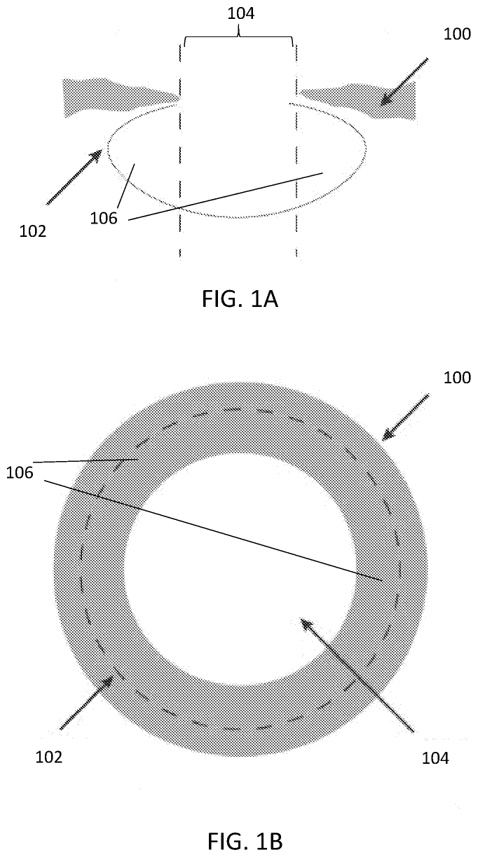

In this regard, FIG. 1A is a diagram of the side view of the anatomy of an eye. The iris 100 blocks most of the lens outer circumference 102 from vision. The area 104 between the lines shown in FIG. 1A is what is visible to the surgeon, from a side view. This figure helps illustrate one problem recognized by the present Applicant of the iris blocking vision of equatorial regions 106 which are outside lines the lens outer circumference.

FIG. 1B is a diagram of the top view of an eye. The lens outer circumference 102 is blocked by the iris 100. The portion 104 inside the iris 100 (corresponding to the area between lines in FIG. 1A) is what is visible to the surgeon. This figure helps illustrate one problem recognized by the present Applicant of the iris blocking vision of the lens outer circumference 102 and equatorial regions 106.

According to certain general aspects, therefore, the present embodiments aim to remedy this problem among others by allowing a user to remove lens material, including but not limited to cortical material and lens epithelial cells, from all regions of the capsular bag, including, but not limited to the anterior, posterior, and equatorial regions of the capsular bag, without a dependency on visualizing the lens material. In one embodiment, the lens material may become mobilized and float to an area in the capsular bag, the area visible by a user of a tool according to embodiments, such that a user can remove the lens material. In alternate embodiments, the user may not visualize the lens material before the cortical material is removed.

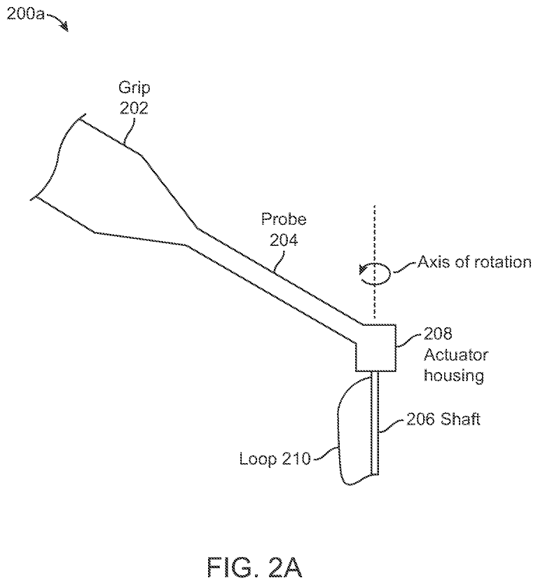

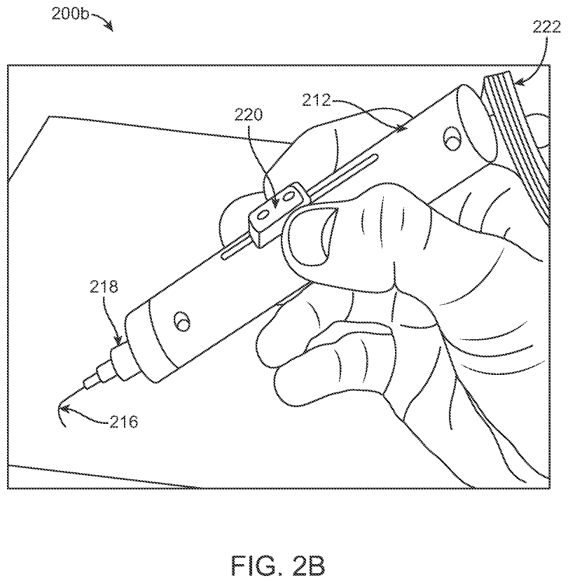

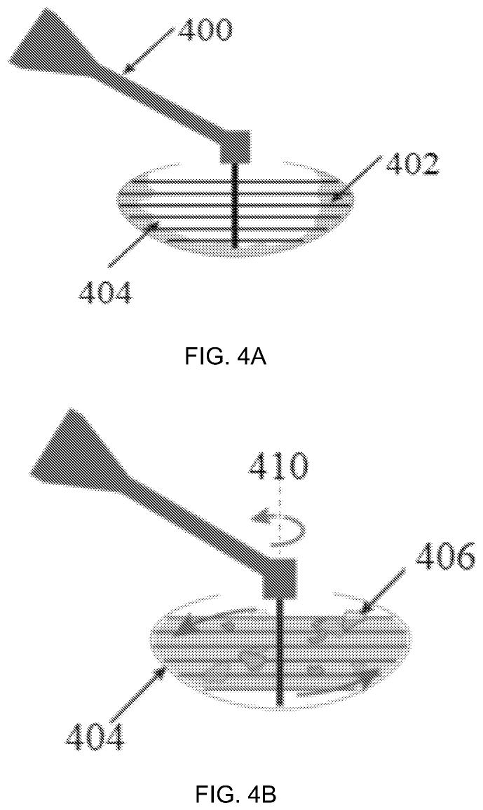

In accordance with these and other aspects, FIG. 2A illustrates an example of a tool used to mobilize lens material from the capsular bag according to embodiments. The tool 200a includes components such as a grip 202, a probe 204, a shaft 206, actuator housing 208, and a flexible loop 210. In some embodiments, one or more components may be added to, or omitted from tool 200a. The loop 210 is an example of an end-effector. Other end-effectors include bristles (e.g., FIGS. 4A-4B). In this example, loop 210 has ends respectively connected to a top and bottom of the shaft 206. Disposed at the top of the shaft 206 is actuator housing 208. The actuator housing houses an actuator that allows the loop 210 to spin around an axis of rotation via the shaft 206. Loop 210 spins around the axis of rotation to brush the inner wall of the capsular bag. The anterior, posterior, and equatorial regions of the capsular bag are effectively polished. The loop is a flexible loop of thin plastic or similar material (e.g., vinyl, fiber, metal, etc.) that changes shape to conform to the shape of the capsular bag as it brushes against the inner wall of the capsular bag.

Tool 200a may receive power through batteries housed inside the tool, through connections to an external power device, or through other methods of delivering power. Power may be used to spin the actuator, or for irrigation and aspiration as discussed further below. The placement of the power delivery system in the tool may be anywhere inside or alongside the tool such that it does not interfere with a user's operation of the tool.

FIG. 2B is a prototype tool 200b used to mobilize cortical material from the capsular bag, according to an embodiment. The tool 200b includes components such as an electrical connector 222, an outer shell or grip 212, a button 220, actuator housing 218, and shaft 216. In some embodiments, one or more components may be added to, or omitted from tool 200b. The electrical connector 222 connects the tool 200b to an external power supply. The external power supply delivers power to tool 200b such that the tool 200b may spin the actuator housed in the actuator housing 218. The tool 200b is manipulated by a surgeon using the grip 212. The grip 212 may be a rigid material (e.g., plastic), a flexible material, or some combination thereof. The grip 212 may be opaque (as shown) or translucent, or some combination thereof. The surgeon may interact with the push-button 220 to extend the loop (not shown) from a loop housing in shaft 216. When extended, the loop may spin along the axis of rotation via the shaft 216.

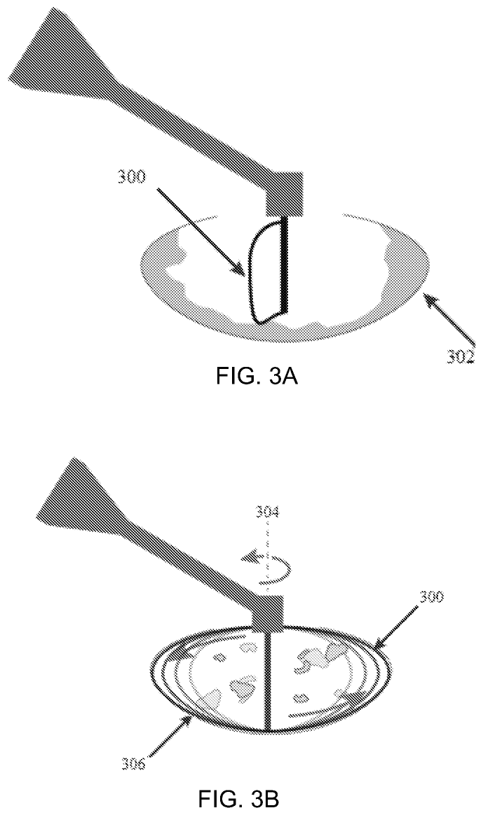

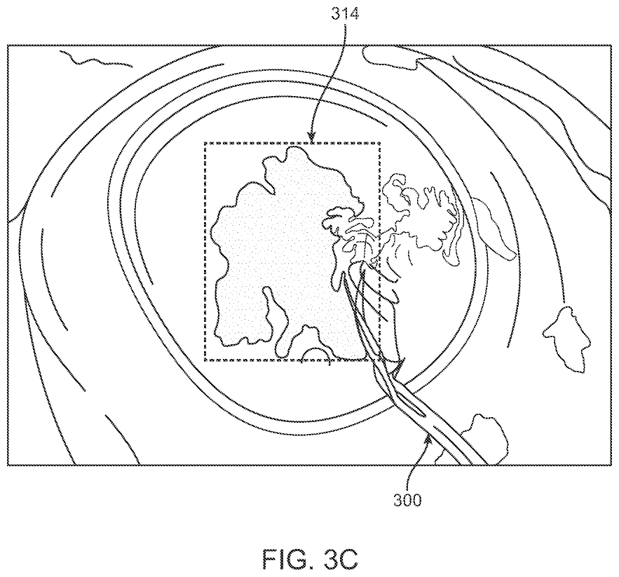

FIG. 3A is a diagram of one embodiment of an example tool operating inside the capsular bag. The lens of the eye is emulsified using known methods in the art (not shown). In one embodiment, the probe, shaft, actuator housing, and flexible loop, are inserted inside the capsular bag. In other embodiments, fewer or additional components of the tool may be inserted inside the capsular bag. The tool may be inserted along the center axis of the capsular bag, after emulsification, through the corneal incision and capsulorhexis, or through other known methods in the art (not shown). Upon entry into the capsular bag, the flexible loop 300 sags under its own weight. When the user commanding the tool activates the tool, by pressing a button, applying pressure at the probe and/or shaft of the tool, pressing a foot pedal, or other methods of activation, the flexible loop 300 begins to spin around an axis of rotation. In one embodiment, cortical material 308 in the equatorial regions of the capsular bag may be hidden by the iris. In this embodiment, the lens equator may be indicated by 302.

FIG. 3B is a diagram of one embodiment of the activated tool inside the capsular bag. Upon activation of the tool, the sagging flexible loop 300 changes shape. Centrifugal force causes the sagging flexible loop 300 to become more rigid and assume a semicircular shape. This semicircular shape, created by the centrifugal forces from the flexible loop spinning around the axis of rotation, is large enough to brush the entire inner wall of the capsular bag including the lens equator. As the flexible loop brushes and polishes the capsular bag, the flexible loop may collide with, for example, cortical material at or near the lens equator, mobilizing the cortical material. The flexible loop 300 spins around the axis of rotation 304 with sufficient force such that the flexible loop 300 mobilizes the lens material in the capsular bag. In one embodiment, the flexible loop can free cortical material inside the capsular bag including at or near the lens equator. In one embodiment, when the flexible loop collides with cortical material, the cortical material may become decomposed into smaller pieces 306 and mobilized. The loop may spin with such revolutions per minute as to mobilize the lens material. The loop does not spin fast enough to rupture the capsular bag, and it does not spin so slowly such that it does not have sufficient force to mobilize the lens material. In one embodiment, the flexible loop spins in one direction for a time period, and subsequently spins in the opposite direction for a time period. In alternate embodiments, the flexible loop spins in one direction.

Although centrifugal force cause the loop to become more rigid, collisions with the lens material may cause the loop to bounce around the capsular bag. In other words, the loop 300 changes shape as it spins along the axis of rotation 304, brushing and polishing all areas of the capsular bag, including the anterior, posterior, and equatorial regions of the capsular bag. The loop also changes shape, back to sagging under its own weight, when the tool is disengaged and centrifugal forces stop acting on the loop. The sagging flexible loop makes the tool easier to enter, and be removed, from the capsular bag.

FIG. 3C is an example of emulsified lens material using a flexible loop, according to an embodiment. As shown in this example, the flexible loop 300 is metal wire. The flexible loop 300 is inserted into a corneal incision and interacts with an intact lens. Upon activation, the lens material becomes emulsified. The emulsified lens material is indicated by indicator 314.

In some embodiments, the end-effector may be or include one or more brush-like bristles. FIG. 4A is a diagram of an alternate embodiment of the tool inside the capsular bag. Similar to FIGS. 3A-3C, the tool may be inserted along the center axis of the capsular bag, after emulsification. In one embodiment, the probe, shaft, actuator housing, and bristles 404, are inserted inside the capsular bag. In other embodiments, fewer or additional components of the tool may be inserted inside the capsular bag. In some embodiments, when a button (or a foot pedal, or a pressure to a particular place on the tool and/or on the end-effector) of the tool is interacted with, bristles 404 may be extended from the shaft of the tool.

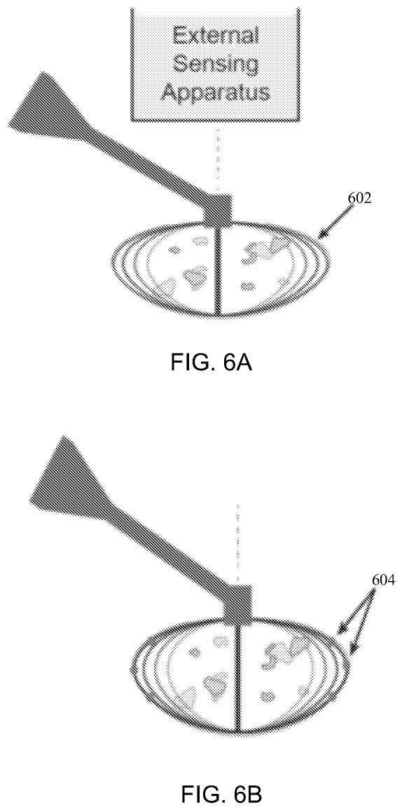

In other embodiments, the bristles 404 may be inserted into the tool at the same time as the shaft is inserted into the tool (e.g., not retracted via the compliant mechanism as described below in connection with FIGS. 6A-6B). The bristles 404 may be flexible and soft such that they do not damage the capsular bag upon entry into the capsular bag. For instance, the bristles may be a flexible and sagging material (e.g. plastic, vinyl, metal, etc. In other embodiments, the bristles 404 may be rigid. Each of the bristles 404 extending from the shaft may be the same length. Additionally or alternatively, each of the bristles 404 may be varying lengths so as to conform to the shape of the capsular bag. In some embodiments, the bristles 404 may extend to the cortical material 402 located at or near the lens outer circumference (i.e. equator). In other embodiments, the bristles 404 may not extend fully to the cortical material 402 located at the lens equator, but the bristles 404 may polish and/or brush the cortical material at or near the lens equator during activation of the tool.

FIG. 4B is a diagram of an alternate embodiment of the activated tool inside the capsular bag. Upon activation of the tool, the bristles 404 may become more rigid and assume a sturdy shape. This sturdy shape, created by the centrifugal forces from the sagging bristles spinning around the axis of rotation 410, is large enough to brush the entire inner wall of the capsular bag including the lens outer circumference (i.e. equator). As the bristles 404 brush and polish the capsular bag, the bristles 404 may collide with, for example, cortical material at the lens equator, mobilizing the cortical material into pieces 406. The bristles 404 spin around the axis of rotation 410 with sufficient force such that the bristles 404 sweep the surface of the lens equator and/or capsular bag, mobilizing the material pieces 406 in the capsular bag. The centrifugal forces of the sweeping bristles 404 spinning around the axis of rotation 410 may, in operation, extend the length of the bristles 404 such that the bristles polish and brush the capsular bag.

Generally, eye lenses are about the same size, around 10 millimeters in diameter and 4 millimeters in length. In one embodiment, when the flexible loop (e.g. FIG. 2A) becomes rigid due to centrifugal force caused by the loop spinning around the axis of rotation, the flexible loop has the same dimensions as the dimensions of the average eye lens. Similarly, when bristles (e.g. FIG. 4A) become sturdy due to centrifugal forces caused by the loop spinning around the axis of rotation, (or upon extending bristles of a tool) the bristles may be the same dimensions as the dimensions of the average eye lens. In alternate embodiments, the loop (or bristles) is larger than the dimensions of the average eye lens to ensure that the loop brushes and polishes all areas of the capsular bag. Alternatively, the loop (or bristles) dimensions can change. For example, the user commanding the tool may lengthen or shorten the dimensions of the loop during the lens material mobilization process. In one embodiment, the shaft can be around 4 to 5 millimeters long. In an alternate embodiment, the shaft dimensions can change during the lens material mobilization process if the user commanding the tool lengthens or shortens the shaft's length.

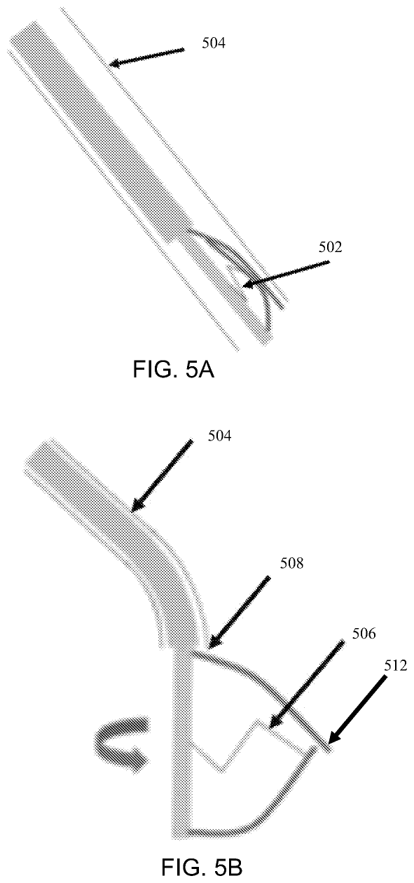

In some embodiments, a compliant mechanism may be added to the end-effector. FIG. 5A is a diagram of one embodiment of a retracted compliant mechanism inside the shaft of the tool. The compliant mechanism 502 is collapsed inside the insertion shaft of the device 504 (e.g., shaft 206 in FIG. 2A). The collapsed compliant mechanism 502 allows for easy entry into the capsular bag and/or easy removal from the capsular bag because the shaft 504 shields the end-effectors from interacting with the capsular bag. The compliant mechanism may retract by employing a spring system, a motorized system, a magnetic system, pressure from the shaft, and the like. The components of the compliant mechanism may be flexible (e.g., plastic) such that the compliant mechanism 502 may be collapsed within the shaft 504.

FIG. 5B is a diagram of one embodiment of an extended compliant mechanism. When the compliant mechanism is extended from the shaft 504 of the device (e.g., activated by a push-button on the tool, a foot pedal, pressure applied to a portion of the tool, and the like) the compliant mechanism may, upon exiting the shaft end 508, assume a shape. As shown, a spring 506 on the compliant mechanism, upon being freed from the shaft 504 at position 508, pushes a loop of material into a flexible loop 512. The newly created flexible loop 512 may be used to clean the capsular bag as discussed herein. The compliant mechanism may retract the spring 506 back into the shaft 504 based on a user input. Upon being removed from the capsular bag, the compliant mechanism may be removed (e.g., disposed of).

The dimensions of the extended compliant mechanism may be variable. The dimensions of the extended compliant mechanism may increase or decrease based on user input. Additionally or alternatively, there may be a variety of compliant mechanisms of pre-determined different dimensions. That is, the compliant mechanisms may be interchangeable.

In some embodiments, the end-effector (e.g., flexible loop of FIGS. 3A-3C, bristles of FIGS. 4A-4B) may be used to sense information regarding the cortical material and/or the end-effector. For instance, the sensed information may include the rotational speed of the end-effector, direction vector of the end-effector, or other information regarding the cortical material (e.g., density, proximity).

FIG. 6A is a diagram of one embodiment of sensing using the end-effector in the capsular bag. The end-effector may be a flexible loop (e.g., FIGS. 3A-3C) made of material that allows the transmission of a signal. That is, the material of the end-effector 602 may facilitate transmission of an optical, magnetic, ultrasound, or other signals. The signal may be processed and displayed or otherwise projected to a surgeon or other user via an external sensing apparatus. The external sensing apparatus may also use the signal to determine information about the end-effector (location, position) and/or cortical material (density).

FIG. 6B is a diagram of an alternate embodiment of sensing using the end-effector in the capsular bag. The end-effector may be a flexible loop (e.g., FIGS. 3A-3C) augmented with one or more sensors 604. The sensors 604 on the flexible loop may enable sensing of information of the end-effector (e.g., speed, acceleration), and/or the cortical material (e.g. density, proximity). In some embodiments, the sensed information may be transmitted to an external sensing apparatus of the tool to be processed and projected to one or more users. In other embodiments, the sensed information may be processed and projected to the user via the tool.

In an alternate embodiment, the tool can assist with irrigation and aspiration. Irrigation and aspiration may be activated via the tool in any combination before the flexible loop spins to mobilize the lens material, after the flexible loop spins to mobilize the lens material, or during the flexible loop spinning and mobilizing the lens material. Those skilled in the art will understand how to integrate known techniques for irrigation and aspiration with a tool according to embodiments after being taught by the present examples.

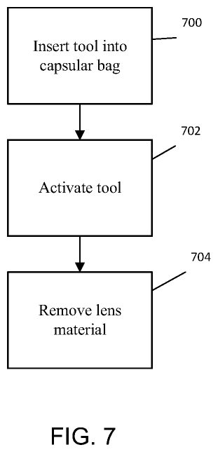

FIG. 7 is a flowchart illustrating exemplary methodology of using an example tool according to an embodiment. In block 700, the tool is inserted into the capsular bag. In one embodiment, the probe, shaft, actuator housing, and flexible loop are inserted inside the capsular bag. In one embodiment, the tool may be inserted through a central corneal incision and capsulorhexis. In one embodiment, the cataract has been emulsified and pieces of the lens material, such as the nucleus, cortical material, and epithelial cells, may remain in the capsular bag. In another embodiment, the tool may be used to break apart the cataract.

In block 702, the user commanding the tool activates the tool. The tool may be activated by pressing a button, applying pressure at the probe and/or shaft of the tool, pressing a foot pedal, or other methods of activation. Upon activation, the end-effector (e.g. flexible sagging loop) spins around the axis of rotation. As the sagging loop spins around the axis of rotation, centrifugal forces cause the loop to change into a semicircular shape and/or conform to the shape of the capsular bag. The loop, in its semicircular shape, brushes all regions of the capsular bag, including, but not limited to the anterior, posterior, and equatorial regions of the capsular bag. The loop spins with sufficient force such that the loop mobilizes the lens material inside the capsular bag. The loop does not spin fast enough to rupture the capsular bag, and it does not spin so slowly such that it does not have sufficient force to mobilize the lens material. In one embodiment, the flexible loop spins in one direction for a time period, and subsequently spins in the opposite direction for a time period. In alternate embodiments, the flexible loop spins in one direction. In one embodiment, the flexible loop spins at one speed in one direction for a time period, and subsequently spins at a second different speed in the one direction for a time period. In alternate embodiments, the flexible loop spins in one direction at one speed only. The loop brushes and polishes all areas of the capsular bag, including, but not limited to, the anterior, posterior, and equatorial regions. The loop spins around the capsular bag such that the loop changes shape, due to centrifugal forces, as it brushes the capsular bag.

In block 704, the lens material is mobilized from the spinning of the flexible loop. The mobilized lens material can subsequently be removed. Removal of the lens material can be through suction, irrigation and aspiration, or other known methods in the art. In one embodiment, the tool can be used to remove the mobilized lens material from the capsular bag.

In additional or alternative embodiments, a handheld tool device is integrated with a connective element that facilitates fast/easy tool-tip exchanges. As examples, this connective element can be a high-strength magnet, a vacuum force, or a mechanical “clasp”. The interchangeable tip can be easily removed by a surgical assistant, the surgeon, or an external automated system (robotic or otherwise).

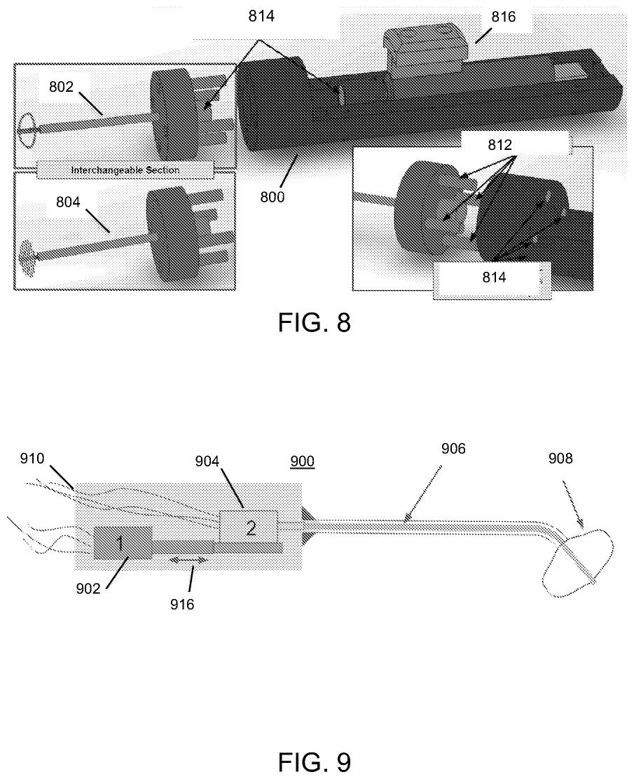

The end-effectors in these and other embodiments can be sterilizable for use in the operating room and/or disposable as single-use items. There are numerous variations of the interchangeable tip design, material type, and function. In FIG. 8, two variations are shown: a tip 802 incorporating a loop-like end-effector (e.g. FIG. 2A) and a tip 804 incorporating a brush-like end-effector (e.g. FIG. 4A), to give two examples. As further shown in the example of FIG. 8, tips 802 and 804 are removably attached to tool body 800 using magnetic steel pins 812 and magnets 814 (e.g. neodymium magnets). When the tips are thus held in place, rotational motion can be translated to the tips 802, 804 via a motor 816 inside the tool body 800. The interchangeable tip concept of FIG. 8 applies to both manual, handheld tool designs as well as robot-mounted designs.

FIG. 9 illustrates an example technique for actuating a tool and end-effector with robotic control and guidance according to embodiments. In this example, tool 900 includes a pair of motors/actuators and can be purely robotic controlled, purely manually controlled, or a combination of both. As shown in FIG. 9, example tool 900 includes actuator/motor 902 that controls the end-effector 908 extension length and/or size by controlling motion in a longitudinal direction 914 of shaft 912. Another motor/actuator 904 controls the end-effector 904's rotational motion (i.e. the cleaning/sweeping motion of tool 900). As shown in this example, this rotational motion is translated to end-effector 904 (e.g. a flexible wire loop) via a torque cable 906 within shaft 912. Wires 910 are shown to illustrate an example of how power and/or control signals can be provided to tool 900 such that tool 900 can be purely robotic controlled, purely manually controlled, or a combination of both.

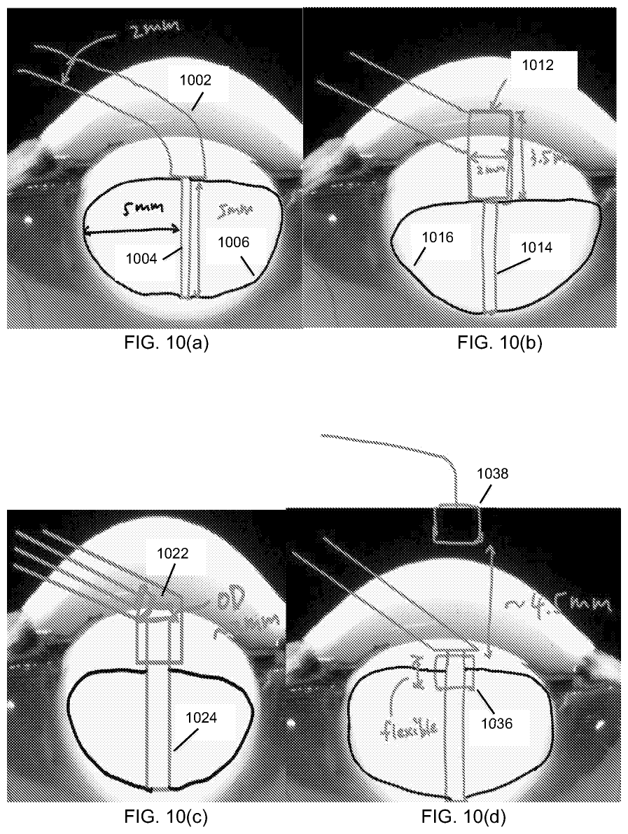

FIGS. 10(a), 10(b), 10(c) and 10(d) illustrate several example techniques to actuate the rotation of an end-effector about an axis of revolution in tools according to embodiments.

FIG. 10 (a) illustrates an example pre-bent tool with a flexible cable inside. This example includes a pre-bent shaft 1002 to access the intraocular anatomy and can contain a torque cable, or other flexible material in the form of a cable, which rotates a rigid rod 1004 hung from the pre-bent shaft 1002 (which thus rotates a flexible loop 1006). Example dimensions in this embodiment include that the rod 1004 extends about 5 mm from the shaft 1002, and the loop 1006 can extend outwards from the shaft by about 5 mm. This design benefits from easy construction and simple geometry. This design may also include the ability to retract the loop, brush, etc. 1006 back into the shaft 1002 for the purpose of increasing safety or entering/exiting the eye.

FIG. 10 (b) illustrates an example tool with a small, miniature motor housed inside. As shown in this example, the actuation device 1012 (e.g., a motor) is housed inside the tool itself and constrains the motion of the end-effector 1016 (e.g. loop, brush, etc.) to a rotation about the central axis of shaft 1014, or any other axis as dictated by the tool geometry. Example dimensions in this embodiment include that the motor 1012 is provided in a housing that has a height of about 3.5 mm and a diameter of about 2 mm.

FIG. 10 (c) illustrates an example tool with a pair or set of bevels or other gears to transmit the rotational angle. As shown in this example, the tool contains a set, or pair, of gears 1022 (beveled, worm, etc.) to transmit power as well as constrain the rotational degree of freedom of shaft 1024 to that which is desired. This design may also include the ability to retract the end-effector 1026 (e.g. loop, brush, etc.) back into the tool for the purpose of increasing safety or entering/exiting the eye. Example dimensions in this embodiment include that the gears 1022 have diameters of about 2 mm.

FIG. 10 (d) illustrates an example tool actuated by an external magnetic field. As shown in this example, the tool contains a magnetic (or other non-contact force) end-effector 1036 (e.g. a shaft and flexible loop). This magnetic component is actuated by a second magnetic (or other non-contact force) component 1032 located outside, but in proximity to (e.g. about 4.5 mm), the eye during operation.

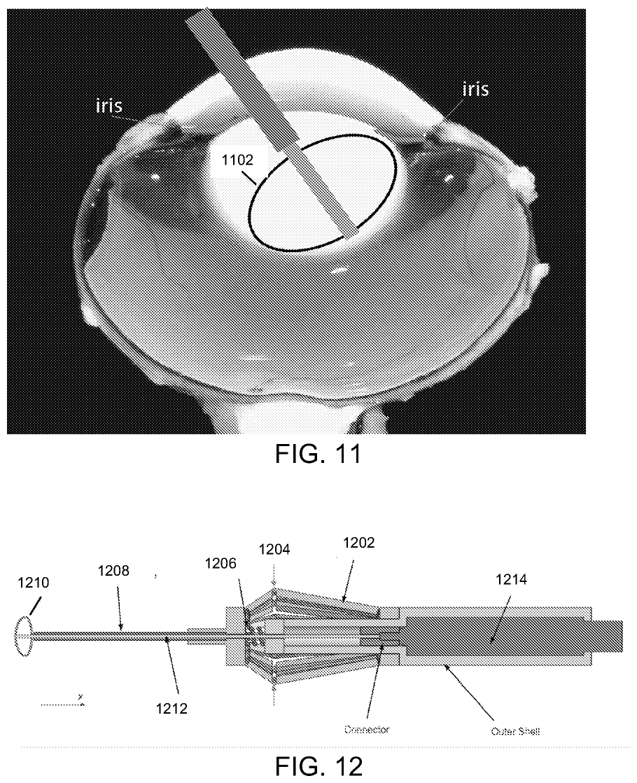

FIG. 11 illustrates another embodiment in which the end-effector portion 1102 of the tool can be straight. As shown, this variation is simple geometrically and physically and can have the ability to retract the end effector back inside the tool for the purpose of entering/exiting the eye to begin/end surgical procedures. The end effector portion can be a loop, a brush, etc. and can rotate about the centerline of a shaft of the tool as an axis of rotation.

FIGS. 12 to 14 illustrate various other examples of a tool according to the present embodiments.

One example variation is illustrated in FIG. 12. It includes a compliant mechanism 1202 that is able to be squeezed by the operator or surgeon. By squeezing this portion (indicated by arrows 1204), a spring 1206 causes a sleeve/needle 1208 to slide forward, which changes the end-effector 1210 size (in this case, a loop). The end-effector 1210 is connected to a rigid rod 1212 which itself is connected to a motor 1214 housed in the base of the device. This motor spins the rigid rod 1212, which in turns spins the end-effector 1210.

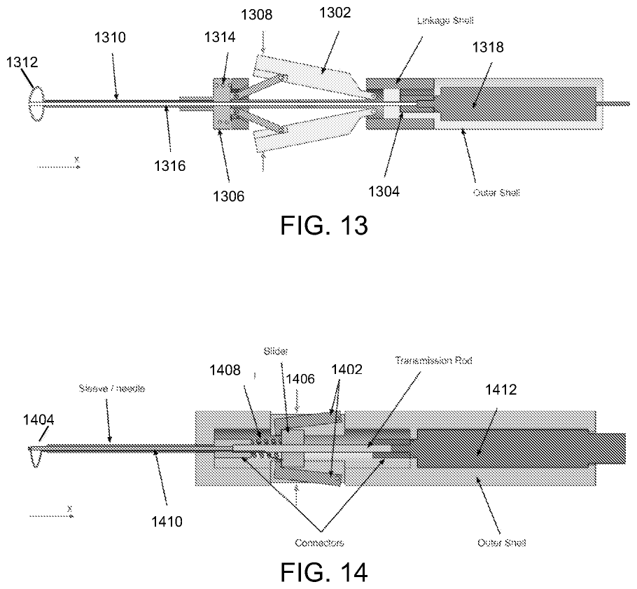

A second example variation is illustrated in FIG. 13. It includes a mechanical linkage 1302 which is connected to the device's base/housing by its rear joints and connector 1304 and to a slider 1306 via its forward joints. By squeezing the linkage 1302 (indicated by arrows 1308), the slider 1306 is pushed forward, which in turn moves the sleeve/needle 1310 forward, thereby shrinking the size of the exposed end effector 1312 (here, a loop). A spring or other return mechanism 1314 housed in the slider reverses this motion. The end-effector 1312 is connected to a rigid rod 1316 which itself is connected to a motor housed in the base of the device. This motor spins the rigid rod, which in turns spins the end effector.

A third example variation is illustrated in FIG. 14. It includes a mechanical linkage (e.g. clamps) 1402 that is used to change the end-effector 1404 length (here, a loop) by changing the length of the exposed end-effector. By pressing down on the linkage 1402 (as indicated by arrows 1406), the end-effector 1404 is drawn inwards. This motion is reversed through the integration of a spring 1408 or other return mechanism. The end-effector is connected to a rigid rod 1410 which itself is connected to a motor 1412 housed in the base of the device. This motor spins the rigid rod, which in turns spins the end effector.

The herein described subject matter sometimes illustrates different components contained within, or connected with, different other components. It is to be understood that such depicted architectures are illustrative, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable and/or physically interacting components and/or wirelessly interactable and/or wirelessly interacting components and/or logically interacting and/or logically interactable components

With respect to the use of plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.).

Although the figures and description may illustrate a specific order of method steps, the order of such steps may differ from what is depicted and described, unless specified differently above. Also, two or more steps may be performed concurrently or with partial concurrence, unless specified differently above. Such variation may depend, for example, on the software and hardware systems chosen and on designer choice. All such variations are within the scope of the disclosure. Likewise, software implementations of the described methods could be accomplished with standard programming techniques with rule-based logic and other logic to accomplish the various connection steps, processing steps, comparison steps, and decision steps.

It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

Further, unless otherwise noted, the use of the words “approximate,” “about,” “around,” “substantially,” etc., mean plus or minus ten percent.

The foregoing description of illustrative embodiments has been presented for purposes of illustration and of description. It is not intended to be exhaustive or limiting with respect to the precise form disclosed, and modifications and variations are possible in light of the above teachings or may be acquired from practice of the disclosed embodiments. It is intended that the scope of the invention be defined by the claims appended hereto and their equivalents.

1. A device for performing surgery on an eye, comprising:
a shaft having an axis of rotation; and
an end-effector coupled to the shaft, wherein the end-effector is configured to mobilize lens material within a capsular bag of the eye when the shaft rotates around the axis of rotation during the surgery.

2. The device of claim 1, wherein the end-effector comprises a flexible loop.

3. The device of claim 2, wherein the flexible loop is configured to extend from the shaft toward equatorial regions of the capsular bag when the shaft rotates around the axis of rotation.

4. The device of claim 2, wherein the flexible loop comprises a plastic material.

5. The device of claim 2, wherein the flexible loop comprises a metal material.

6. The device of claim 1, wherein the end-effector comprises a brush having one or more bristles extending from the shaft.

7. The device of claim 6, wherein the bristles are rigid.

8. The device of claim 1, wherein the end-effector comprises a compliant mechanism that is configured to cause a flexible material to extend away from and retract toward the shaft.

9. The device of claim 1, further comprising sensing elements integrated in the end-effector and configured to sense one or more parameters of motion of the end-effector.

10. A method of mobilizing lens material in a capsular bag of an eye during cataract surgery, comprising:
inserting at least a portion of a tool into the capsular bag, the portion including an end-effector; and
causing the end-effector to spin around an axis of rotation inside the capsular bag.

11. The method of claim 10, wherein causing the end-effector to spin includes rotating the end-effector in first and second directions at first and second times, respectively.

12. The method of claim 10, wherein causing the end-effector to spin includes rotating the end-effector at first and second speeds at first and second times, respectively.

13. A tool having the device of claim 1, wherein the end-effector is removably coupled to a housing having a motor using magnets.

14. A tool having the device of claim 1, further comprising a motor coupled to the shaft.

15. A tool having the device of claim 1, further comprising a set of gears between the shaft and a motor.
